(19) United States
(12) Patent Application Publication
(10) Pub. No.: US 2010/0075405 A1
(43) Pub. Date: Mar. 25, 2010

(54) BIOREACTOR JACKET

(75) Inventors: Scott T. Broadley, Los Angeles, CA (US); Patricia R. Benton, Foothill Ranch, CA (US)

Correspondence Address: KNOBBE MARTENS OLSON & BEAR LLP, 2040 MAIN STREET, FOURTEENTH FLOOR, IRVINE, CA, 92614, US

(73) Assignee: BROADLEY-JAMES CORPORATION, Irvine, CA (US)

(21) Appl. No.: 12/525,300

(22) PCT Filed: Feb. 14, 2008
(86) PCT No.: PCT/US08/54026

§ 371 Date: Jul. 30, 2009

Related U.S. Application Data
(60) Provisional application No. 60/890,158, filed on Feb. 15, 2007.

(51) Int. Cl. C12M 1/38; C12M 1/02; F17D 1/00

(52) U.S. Cl. 435/286.1; 435/289.1; 137/2

(57) ABSTRACT

A bioreactor system (10) is provided for controlling temperature of bioreactor media during processing. The system (10) includes a flexible bag (102) having an inner wall (104) defining an inner volume (105) for holding the cell culture media and an outer wall (106) surrounding at least a portion of the inner wall (104). Together, the inner wall (104) and the outer wall (106) form a jacket volume (107) which can hold a temperature control fluid.

### BACKGROUND OF THE INVENTION

1. Field of the Invention

The present invention relates to a disposable bioreactor heating and cooling system and methods of controlling temperature in a disposable bioreactor bag.

2. Description of the Related Art

Bioreactors (also referred to as fermenters) include containers used for fermentation, enzymatic reactions, cell culture, tissue engineering, and food production, as well as in the manufacture of biologicals, chemicals, biopharmaceuticals, microorganisms, plant metabolites, and the like. Bioreactors vary in size from benchtop fermenters to stand-alone units of various sizes. Small-scale bioreactors have also been developed which comprise pre-sterilized, disposable flexible bags configured to hold cell culture media.

As cell fermentation processes are highly sensitive to temperature variations, bioreactor systems require temperature-control mechanisms to maintain uniformity and stability of temperature throughout the bioreactor medium. Control mechanisms exist which comprise a heating blanket configured to surround a bioreactor bag. Such a heating blanket may comprise, for example, a silicon rubber blanket with wires running through it. These resistive heat blankets, however, are capable of heating the bioreactor medium but cannot cool the medium.

One method of providing both heating and cooling capability in a disposable bioreactor system is to provide a double-walled rigid vessel to support the bioreactor bag. The double walls of the vessel are filled with a fluid, such as water, which is circulated around the bag and pumped through an external heating or cooling device. Double-walled rigid vessels such as these, however, can be extremely expensive.

### SUMMARY OF CERTAIN EMBODIMENTS

The system, method, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of Certain Embodiments” one will understand how the features of this invention provide advantages over other bioreactor systems.

In a first aspect of the invention, a bioreactor bag is provided for affecting the temperature of media held therein. The bag comprises a flexible inner wall defining an inner volume for holding the media and a flexible outer wall surrounding at least a portion of the inner wall. The inner wall and the outer wall together define an outer volume for holding a temperature control fluid therein, the outer volume being separate from the inner volume. In an embodiment of the first aspect, the bag also includes an entrance port configured to receive the temperature control fluid into the outer volume and an exit port configured to exhaust the fluid from the outer volume. In an embodiment of the first aspect, the fluid is a liquid. In another embodiment, the fluid is a gas. In a further embodiment, the inner volume of the bioreactor bag has a capacity of between about 100 milliliters and about 5000 liters. In another embodiment, the outer volume of the bioreactor has a capacity between about 1 percent and about 50 percent of the inner volume capacity. In another embodiment, the inner wall and the outer wall comprise plastic. In a further embodiment, the outer volume entirely encircles the inner volume when the inner volume is at least partially filled with media and the outer volume is at least partially filled with fluid. In a still further embodiment, the outer volume extends below the inner volume when the inner volume is at least partially filled with media and the outer volume is at least partially filled with fluid. In another embodiment, the system further comprises a temperature control module configured to affect the temperature of the fluid in the outer volume. In such an embodiment, the temperature control module can comprise a heat exchanger disposed outside the outer volume. Additionally or alternatively, the temperature control module can comprise a temperature adjusting element disposed in contact with the outer volume. The temperature adjusting element can extend into the outer volume. The temperature adjusting element can be a resistive heating element, a cold finger, or a hot finger.

In a second aspect of the invention, a bioreactor system is provided for affecting the temperature of media. The system includes a flexible jacket bag configured to hold a temperature control fluid and configured to at least partially surround a bioreactor bag when the bioreactor bag is at least partially filled with media. The jacket bag has an inlet port and an outlet port, the inlet port being configured to receive the temperature control fluid into the jacket bag from a temperature control mechanism, the outlet port configured to exhaust the fluid from the jacket bag. In an embodiment of the second aspect, an interior wall of the jacket bag is configured to contact an outer wall of the bioreactor bag when the jacket bag is at least partially filled with the temperature control media. In another embodiment, the system further includes the bioreactor bag. In another embodiment, the system further includes the temperature control mechanism.

In a third aspect, a bioreactor bag is provided which comprises first flexible means for holding media in a sterile environment and second flexible means for holding a temperature control fluid separate from but adjacent to the media. The second flexible means at least partially surrounds the first flexible means and comprises means for receiving and exhausting the temperature control fluid.

In a fourth aspect, a method of regulating the temperature of media during processing is provided. The method comprises providing a bioreactor bag comprising a flexible inner wall defining an inner volume for holding the media and a flexible outer wall surrounding at least a portion of the inner wall, the inner wall and the outer wall together defining an outer volume for holding a temperature control fluid therein, the outer volume being separate from the inner volume. The method also comprises filling the outer volume at least partially with the temperature control fluid. In an embodiment of the fourth aspect, the method further comprises adjusting the temperature of the temperature control fluid.

In a fifth aspect, a method of regulating temperature in a flexible bioreactor bag is provided. The bioreactor bag has a first flexible surface configured to hold media and a second flexible surface surrounding at least a portion of the first surface, the first surface and the second surface defining a cavity therebetween. The method comprises providing fluid into the cavity formed between the first surface of the bioreactor bag and the second surface of the bioreactor bag through an entrance port, exhausting fluid from the cavity through an exit port, and affecting the temperature of media held by the first flexible surface by the temperature of the fluid provided to the cavity. In an embodiment of the fifth aspect, the method further comprises controlling the temperature of the fluid provided to the flexible bioreactor bag. In such an embodiment, the method can also comprise sensing the temperature of media held by the first flexible surface and controlling the temperature of the fluid provided to the flexible bioreactor bag based on the sensed temperature.

### BRIEF DESCRIPTION OF THE DRAWINGS

FIG. 1A shows a side elevation view of a bioreactor system according to an embodiment of the present invention.

FIG. 1B shows a side elevation view of a bioreactor system according to another embodiment of the present invention.

FIG. 2 shows a side elevation view of a bioreactor system according to another embodiment.

FIG. 3 shows a cross-sectional view of the bioreactor system of FIG. 1A taken along line 3-3 of FIG. 1A.

FIG. 4 shows a perspective view of a bioreactor system according to a further embodiment of the present invention.

FIG. 5 shows a cross-sectional view of the bioreactor system of FIG. 4 taken along line 5-5 of FIG. 4 and resting in a bioreactor vessel.

FIG. 6 shows a basic process diagram illustrating a method of regulating temperature in a bioreactor bag according to an embodiment.

FIG. 7 shows a basic process diagram illustrating a method of regulating temperature in a bioreactor bag according to another embodiment.

### DETAILED DESCRIPTION OF THE PREFERRED EMBODIMENTS

The following detailed description is directed to certain specific embodiments of the invention. However, the invention can be embodied in a multitude of different ways. In this description, reference is made to the drawings wherein like parts are designated with like numerals throughout.

Various embodiments of the invention provide for systems and methods of regulating temperature in a disposable multi-wall flexible or semi-flexible bioreactor container or bag. Such “bags” are sometimes referred to herein as a “single use bioreactor” or “bioreactor bag.” Embodiments of the invention include a flexible bioreactor bag having an inner wall that defines an inner volume for holding the cell culture media, and an outer wall that surrounds at least a portion of the inner wall. Together, the inner wall and the outer wall form a temperature control jacket configured to hold a temperature control fluid. The built-in temperature control jacket provides a convenient and inexpensive way of regulating the temperature of the cell culture media held in the inner volume.

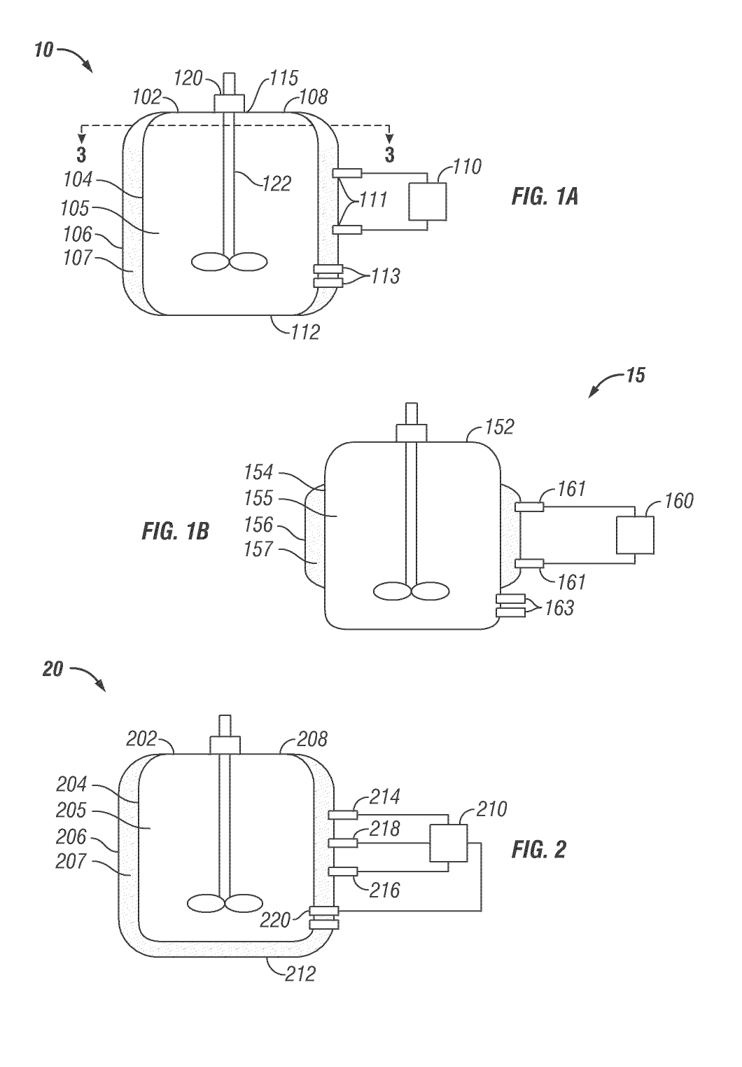

Referring to FIG. 1A, in one embodiment a bioreactor system 10 includes a flexible bioreactor bag 102 having an interior wall 104 and an exterior wall 106 which define a cavity therebetween. The interior wall 104 defines an interior volume 105 of the bag 102, and is configured to receive and hold a fluidic medium such as a cell culture medium (“media”). The bag 102 may further be configured to provide a sterile environment, for example, for culturing cells or other organisms, for microbial fermentation, for food production, etc. The bag 102 may comprise a single layer, or two or more layers of flexible or semi-flexible material capable of containing media and/or a cooling fluid. The material used for particular application may vary depending on size, strength and volume requirements of the application.

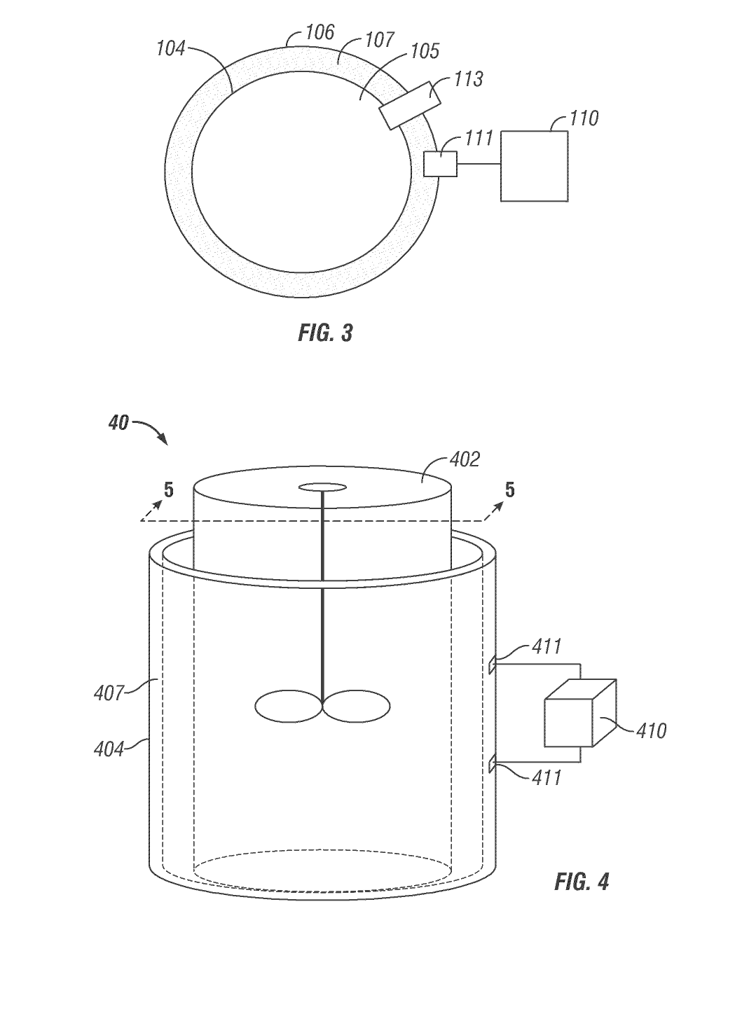

The interior wall 104 and the exterior wall 106 together define an outer or jacket volume 107, which, as illustrated in FIG. 3, may surround all, or a portion, of the interior volume 105. In some embodiments, the jacket volume 107 does not completely surround the top of the interior volume 105, and/or the bottom of the interior volume. For example, FIG. 1A illustrates a typical embodiment where the jacket volume 107 encircles the sides of the interior volume 105. FIG. 1B illustrates a bioreactor system 15 according to a further embodiment, including a bioreactor bag 152 comprising an interior wall 154 and an exterior wall 156. The interior wall 154 defines an interior volume 155 of the bag 152. The exterior wall is configured as a “belly band” which encircles a middle portion of the interior wall 154 to define a jacket volume 157. In other embodiments (FIG. 2 illustrating one such embodiment) the jacket volume may also underlie at least a portion of the bottom of the interior volume and/or at least a portion of the top of the interior volume. In other embodiments, the jacket volume may be segregated into sections, and configured such that only certain sections are in fluid connection with a provided heating or cooling fluid. Such an embodiment may be advantageous where circulating a smaller amount of fluid in the jacket volume is desired. In some embodiments, the interior volume may have a capacity of between about 100 milliliters and about 5000 liters. In some exemplary embodiments, the jacket volume may have a capacity of between about 1% and about 50% of the interior volume.

Referring again to FIG. 1A, the bioreactor system assembly 10 further includes a temperature control mechanism 110 configured to regulate the temperature of the fluid circulating through the jacket volume 107. The temperature control mechanism 110 can be configured to provide a heating and/or cooling fluid to the jacket volume 107.

With continued reference to FIG. 1A, the interior wall 104 of the bag 102 may comprise one or more sheets of flexible plastic, fused together at one or more seams to create the interior volume 105 separate from the jacket volume 107. When in use, the interior volume 105 can be hermetically sealed from the jacket volume 107 and the exterior of the bag 102 by the seams and seals around any bag openings. When filled with medium and air, the bag 102 may have a generally cylindrical, or lenticular shape. The exterior wall 106 may also comprise one or more sheets of plastic, fused together at one or more seams, and also fused with the interior wall 104 at one or more seams to create the jacket volume 107. In some embodiments and as illustrated in FIG. 1A, the interior wall 104 and the exterior wall 106 may converge at a ceiling 108 and a floor 112, so that the jacket volume 107 has a generally tubular shape.

The ceiling 108 may have an opening 115 configured to closely receive and seal with an agitation assembly 120. In some embodiments, the agitation assembly 120 includes portion of an agitation system or an agitation drive unit, coupled with an agitator 122. The agitator 122 may extend from into the interior volume 105, and may be configured to stir or agitate the cell medium during the fermentation process.

The exterior wall 106 may include openings or ports 111 configured to allow fluid to pass from the jacket volume 107 to the temperature control mechanism 110, and from the temperature control mechanism 110 back to the jacket volume 107. Any fluid capable of heating or cooling the interior volume may be used, including, but not limited to, air and water. The assembly 10 may further include ports 113 passing through both the exterior wall 106 and the interior wall 104. In the embodiment illustrated in FIG. 1B, the exterior wall 156 includes ports 161 which communicate with a temperature control mechanism 160. Ports 163 for probes or other sensors are provided in the bag 152 outside the region of the “belly band” jacket volume 157. The ports 113, 163 may be configured to allow passage of probes, such as pH probes, dissolved oxygen probes, or temperature sensors (not shown) through the interior wall 104 into the medium contained within the bag 102. The ports 113, 163 may comprise, for example, Pall Kleenpak™ connectors, allowing for sterile introduction of such probes into the cell medium. In embodiments comprising temperature sensors, the temperature sensors can be configured to provide feedback to the temperature control mechanism.

Embodiments of the invention can also be used with different agitation systems, such as those illustrated in FIGS. 1-4, or in bioreactor bags designed to be agitated with wave-motion or rocking motion, for example, multi-wall “pillow-shaped” bioreactor bags.

### Temperature Control Mechanism

The temperature control mechanism 110 may, for example, be a heat exchanger or any other suitable means for adjusting temperature of the fluid circulating through the jacket volume 107. The temperature control mechanism 110 may additionally comprise a pump mechanism (not shown) configured to continuously or intermittently re-circulate fluid through the jacket volume 107.

In an embodiment illustrated in FIG. 2, a bioreactor system 20 may include a bioreactor bag 202 having an interior wall 204 and an exterior wall 206. The interior wall 204 of the bag 202 may comprise one or more sheets of plastic, fused together at one or more seams to create the interior volume 205. The exterior wall 206 may also comprise one or more sheets of plastic, fused together at one or more seams, and also fused with the interior wall 204 at one or more seams to create the jacket volume 207. As shown in the figure, the interior wall 204 and the exterior wall 206 may be fused together at a ceiling 208. The walls 204, 206 may otherwise remain separate from each other, including at the floor 212, to define a jacket volume 207 surrounding and underlying the interior volume 205.

The embodiment in FIG. 2 also illustrates a fluid supply unit 210 which is configured to circulate a temperature control fluid through the jacket volume 207. Ports 214, 216 can be provided in the bag 202 to receive and exhaust, respectively, the fluid from the jacket volume 207. A pump (not shown), typically provided as port of the fluid supply system 210, can be configured to continuously or intermittently circulate the fluid through the jacket volume 207. The system 20 can include one or more temperature adjustment elements 218 configured to adjust the temperature of the fluid in the jacket volume 207. The temperature adjustment element 218 can extend into the jacket volume 207 at a side portion of the bag 202, as shown in the figure, or at a top or bottom portion of the bag 202. The exterior wall 206 can include one or more pockets or ports to receive the temperature adjustment element 218. Additionally or alternatively, the temperature adjustment element 218 can contact the exterior wall 206 in any other configuration suitable for transferring heat to and/or from the fluid in the jacket volume 207. The temperature adjustment element 218 can comprise, for example, a resistive heating element, a cold finger, or a hot finger. One or more temperature sensors 220 can also be provided which are configured to sense the temperature of the media held in the interior volume of the bag. The temperature sensor 220 can be configured to provide feedback to the fluid supply unit 210. Additionally or alternatively, a temperature adjustment element, such as a heat exchanger, can be operatively coupled to the fluid supply unit and configured to regulate the temperature of the fluid.

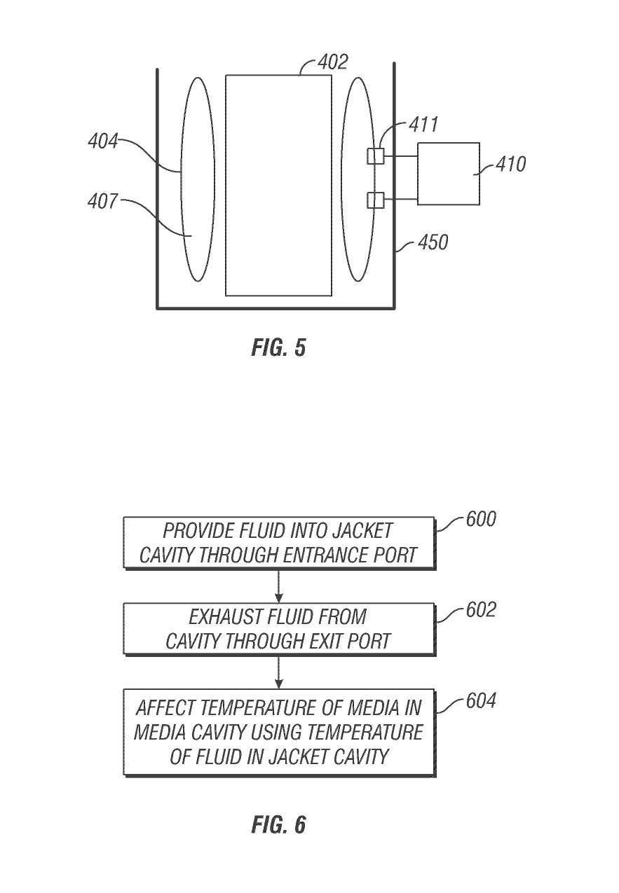

In yet another embodiment, as illustrated in FIGS. 4 and 5, a bioreactor system 40 includes a flexible bioreactor bag 402, a separate jacket bag 404, and a temperature control mechanism 410. FIG. 4 shows a perspective view of a bioreactor system 40, and FIG. 5 shows a cross-sectional view of the bioreactor system 40. The bioreactor bag 402 may be configured to receive and support a cell culture medium. The jacket bag 404 may comprise one or more sheets of flexible plastic, fused together at one or more seams so as to define a jacket volume 407. The jacket bag 404 may be configured to surround the filled or partially filled bioreactor bag 402. Accordingly, the jacket bag may have a generally tubular or cylindrical shape. The jacket bag 404 may further be configured to receive a heating or cooling fluid flowing through the jacket volume 407. The jacket bag may have one or more openings or ports 411 configured to allow fluid to pass from the jacket volume 407 to the temperature control mechanism 410, and from the temperature control mechanism 410 back to the jacket volume 407.

With continued reference to FIG. 5, the jacket bag 404 may be configured to stand in a bioreactor vessel 450, such as a glass or metal bioreactor vessel, when the jacket bag 404 is filled or partially filled with fluid. To achieve this configuration the jacket bag 404 may be provided with reinforcements (not shown) at a bottom portion of the jacket bag 404. The reinforcements may comprise, for example, an additional layer of rigid or semi-rigid plastic fused with the jacket bag 404. The jacket bag 404 may also be configured to support and maintain the position of the bioreactor bag 402 within the vessel 450. The jacket bag 404 may further be disposable or re-usable. Although shown with the bioreactor bag 402 and the jacket bag 404 spaced apart to illustrate that the bags 402, 404 are separate, the outer wall of the bag 402 can of course contact the inner wall of the jacket bag 404.

One advantage of a jacket bag is that the size and thickness of the jacket bag can be selected to allow placement and use of a bioreactor bag of a given capacity in a rigid bioreactor vessel of a greater capacity. Furthermore, a jacket bag may be completely filled or only partially filled as required by the sizes and capacities of the provided bioreactor bag and bioreactor vessel. Because the jacket bag is flexible, embodiments of the invention also desirably allow placement and use of a bioreactor bag having a given cross-sectional shape, such as a lenticular shape, in a vessel having a cylindrical shape. Embodiments of the invention thus allow for greater flexibility of operation and development using fewer rigid bioreactor vessels. Furthermore, these and other embodiments also desirably allow for precise adjustment of temperature in a bioreactor bag, whether heating or cooling is required.

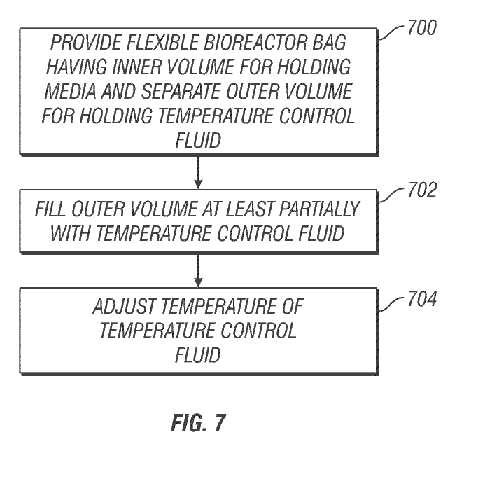

Embodiments of the invention also comprise methods of regulating temperature in a bioreactor bag. With reference now to FIG. 6, according to one embodiment, the method includes providing fluid into a jacket cavity formed between two flexible surfaces or walls of a bioreactor bag. Cell culture media is held in a separate media cavity formed by one of the surfaces of the bioreactor bag. At process step 600, the fluid is provided into the jacket cavity through an entrance port in the bioreactor bag. At process step 602, the fluid is exhausted from the jacket cavity through an exit port. At process step 604, the temperature of the media held in the media cavity is affected by the temperature of the fluid provided in the jacket cavity. According to another embodiment, illustrated in FIG. 7, a method of regulating the temperature of media during processing includes a process step 700 of providing a specially configured bioreactor bag. The bioreactor bag has a flexible inner wall defining an inner volume for holding the media, and a flexible outer wall surrounding at least a portion of the inner wall. Together, the inner wall and the outer wall define an outer volume for holding a temperature control fluid. The outer volume is separate from the inner volume. The method also includes a process step 702 of at least partially filling the outer volume with the temperature control fluid. The method can also include the process step 704 of adjusting the temperature of the temperature control fluid.

Various modifications to these examples may be readily apparent to those skilled in the art, and the generic principles defined herein may be applied to other examples without departing from the spirit or scope of the novel aspects described herein. For example, aspects described in connection with particular embodiments can be combined with aspects of other embodiments. Thus, the scope of the disclosure is not intended to be limited to the examples shown herein but is to be accorded the widest scope consistent with the principles and novel features disclosed herein.

What is claimed is:

1. A bioreactor bag for affecting the temperature of media held therein, the bag comprising:
a flexible inner wall defining an inner volume for holding the media; and
a flexible outer wall surrounding at least a portion of the inner wall, the inner wall and the outer wall together defining an outer volume for holding a temperature control fluid therein, the outer volume being separate from the inner volume.

2. The bioreactor bag of claim 1, further comprising:
an entrance port configured to receive the temperature control fluid into the outer volume; and
an exit port configured to exhaust the fluid from the outer volume.

3. The bioreactor bag of claim 1, wherein the fluid is a liquid.

4. The bioreactor bag claim 1, wherein the fluid is a gas.

5. The bioreactor bag of claim 1, wherein the inner volume of the bioreactor bag has a capacity of between about 100 milliliters and about 5000 liters.

6. The bioreactor bag of claim 1, wherein the outer volume of the bioreactor has a capacity between about 1 percent and about 50 percent of the inner volume capacity.

7. The bioreactor bag of claim 1, wherein the inner wall and the outer wall comprise plastic.

8. The bioreactor bag of claim 1, wherein the outer volume entirely encircles the inner volume when the inner volume is at least partially filled with media and the outer volume is at least partially filled with fluid.

9. The bioreactor bag of claim 1, wherein the outer volume extends below the inner volume when the inner volume is at least partially filled with media and the outer volume is at least partially filled with fluid.

10. A bioreactor system comprising the bioreactor bag of claim 1, the system further comprising a temperature control module configured to affect the temperature of the fluid in the outer volume.

11. The bioreactor system of claim 10, wherein the temperature control module comprises a heat exchanger disposed outside the outer volume.

12. The bioreactor system of claim 10, wherein the temperature control module comprises a temperature adjusting element disposed in contact with the outer volume.

13. The bioreactor system of claim 12, wherein the temperature adjusting element extends into the outer volume.

14. The bioreactor system of claim 12, wherein the temperature adjusting element is a resistive heating element.

15. The bioreactor system of claim 12, wherein the temperature adjusting element is a cold finger.

16. The bioreactor system of claim 12, wherein the temperature adjusting element is a hot finger.

17. A bioreactor system for affecting the temperature of media, the system comprising:
a flexible jacket bag configured to hold a temperature control fluid and configured to at least partially surround a bioreactor bag when the bioreactor bag is at least partially filled with media, the jacket bag comprising
an inlet port configured to receive the temperature control fluid into the jacket bag from a temperature control mechanism, and
an outlet port configured to exhaust the fluid from the jacket bag.

18. The bioreactor system of claim 17, wherein an interior wall of the jacket bag is configured to contact an outer wall of the bioreactor bag when the jacket bag is at least partially filled with the temperature control media.

19. The bioreactor system of claim 17, further comprising the bioreactor bag.

20. The bioreactor system of claim 17, further comprising the temperature control mechanism.

21. A bioreactor bag comprising:
first flexible means for holding media in a sterile environment; and
second flexible means for holding a temperature control fluid separate from but adjacent to the media, the second flexible means at least partially surrounding the first flexible means, the second flexible means comprising means for receiving and exhausting the temperature control fluid.

22. A method of regulating the temperature of media during processing, the method comprising:
providing a bioreactor bag comprising a flexible inner wall defining an inner volume for holding the media and a flexible outer wall surrounding at least a portion of the inner wall, the inner wall and the outer wall together defining an outer volume for holding a temperature control fluid therein, the outer volume being separate from the inner volume; and
filling the outer volume at least partially with the temperature control fluid.

23. The method of claim 22, further comprising adjusting the temperature of the temperature control fluid.

24. A method of regulating temperature in a flexible bioreactor bag having a first flexible surface configured to hold media and a second flexible surface surrounding at least a portion of the first surface, the first surface and the second surface defining a cavity therebetween, the method comprising:
providing fluid into the cavity formed between the first surface of the bioreactor bag and the second surface of the bioreactor bag through an entrance port;
exhausting fluid from the cavity through an exit port; and
affecting the temperature of media held by the first flexible surface by the temperature of the fluid provided to the cavity.

25. The method of claim 24, further comprising controlling the temperature of the fluid provided to the flexible bioreactor bag.

26. The method of claim 25, the method further comprising:
sensing the temperature of media held by the first flexible surface; and
controlling the temperature of the fluid provided to the flexible bioreactor bag based on the sensed temperature.
